(19) United States
(12) Patent Application Publication
(10) Pub. No.: US 2015/0285804 A1
(43) Pub. Date: Oct. 8, 2015

(54) DIAGNOSTIC METHOD FOR COLORECTAL CANCER

(71) Applicant: ORIGIN SCIENCES LIMITED, London (GB)

(72) Inventor: Jodie Louise Booth, Cambridgeshire (GB)

(21) Appl. No.: 14/435,108

(22) PCT Filed: Oct. 11, 2013
(86) PCT No.: PCT/GB2013/052654

§ 371 Date: Apr. 10, 2015

(30) Foreign Application Priority Data
Oct. 12, 2012 (GB) 1218341.4

(51) Int. Cl. G01N 33/574

(52) U.S. Cl. CPC G01N 33/57419

(57) ABSTRACT

The present invention relates to a method of diagnosing patients with colorectal cancer by quantifying the level of one or more proteins, for example immunoglobulins such as IgM, IgG, IgA (total and/or secretory IgA), in a sample of the colorectal mucosa. The invention also relates to methods of monitoring efficacy of colorectal cancer therapy and to kits for diagnosing colorectal cancer by using the method described herein.

### FIELD OF THE INVENTION

The present invention relates to a method of diagnosing patients with colorectal cancer by quantifying the level of one or more proteins in a sample of the colorectal mucosa. The invention also relates to methods of monitoring efficacy of colorectal cancer therapy and to kits for diagnosing colorectal cancer by using the method described herein.

### BACKGROUND OF THE INVENTION

Colorectal cancer is thought to be the third most common cancer in the world, with almost 60% of cases being diagnosed in the developed world. It can be difficult to diagnose, particularly because symptoms are often left unnoticed until a tumor is well advanced. However, early diagnosis of colorectal cancer is important in order to improve a patient's chances of survival.

Typical methods of diagnosis of colorectal cancer include fecal occult blood testing, sigmoidoscopy and colonoscopy. Fecal occult blood testing can give a general indication of bleeding in the bowel, but it is not able to provide an early diagnosis of colorectal cancer or indicate the presence of polyps which may develop into colorectal cancer. Sigmoidoscopy and colonoscopy techniques are usually used to screen for colorectal cancer, but these methods can be time consuming and expensive.

Individuals with Inflammatory Bowel Disease, including Ulcerative Colitis and Crohn's Disease, have an increased risk of developing colorectal cancer. Therefore, patients with these disorders need to be screened regularly in order to increase the chance of diagnosing colorectal cancer early.

WO2010/109196 describes a method of diagnosing and monitoring cancer using non-IgG immunoglobulins to bind carbohydrate-containing antigens in serum samples.

An object of the invention is to provide a simplified and accurate method of diagnosing colorectal cancer.

### SUMMARY OF THE INVENTION

According to a first aspect of the invention, there is provided an in vitro method of diagnosing colorectal cancer in a patient, which comprises the steps of:
(a) quantifying the level of one or more proteins in a sample of the colorectal mucosa obtained from the patient; and
(b) comparing the level of the one or more proteins quantified in step (a), with the level of the one or more proteins in one or more control samples, such that where the control sample is from a healthy subject, a difference in the level of the one or more proteins is indicative of a diagnosis of colorectal cancer, and where the control sample is from a subject with colorectal cancer, a similarity in the level of one or more proteins is indicative of a diagnosis of colorectal cancer.

According to a further aspect of the invention, there is provided an in vitro method of diagnosing colorectal cancer in a patient, comprising:
(a) quantifying the level of one or more proteins in a sample of the colorectal mucosa obtained from the patient; and
(b) comparing the levels of the one or more proteins in the sample with the amounts present in a sample obtained from the patient on a previous occasion, such that a change in the level of the one or more proteins in the sample is indicative of a diagnosis of colorectal cancer in the patient.

According to a further aspect of the invention, there is provided an in vitro method of monitoring efficacy of a therapy for colorectal cancer in a patient having such a disorder or suspected of having such a disorder, comprising:
(a) quantifying the level of one or more proteins in a sample of the colorectal mucosa obtained from the patient; and
(b) comparing the levels of the one or more proteins in the sample with the amounts present in a sample obtained from the patient on a previous occasion, such as prior to commencement of therapy, such that a difference in the level of the one or more proteins in the sample is indicative of a beneficial effect of the therapy.

According to a further aspect of the invention, there is provided a kit for use in the methods as defined herein, comprising a biosensor capable of detecting and/or quantifying the one or more proteins from the sample of the colorectal mucosa.

### BRIEF DESCRIPTION OF THE FIGURES

FIG. 1: Standard curves for the four different antibody assays.

Example standard curves were generated from duplicate values of standards ranging from 0-500 ng/ml and analysed using GraphPad Prism (v5.04).

FIG. 2: Detection of antibodies in 50× (A) and 100× diluted (B) human colorectal mucosal samples.

Two samples were collected from the rectal mucosa of patients subsequently confirmed by colonoscopy to have no specific abnormality of the colon, and tested for the presence of IgA, IgG and IgM antibodies. Two dilutions of sample were tested to permit determination of the concentration of the antibodies using standard curves similar to those illustrated in FIG. 1. Mean of duplicate values are plotted. Error bars indicate 95% confidence interval.

FIG. 3: Comparison of the average total immunoglobulin (Ig) concentration in sample groups.

IgA, IgG and IgM antibodies were detected in various sample groups by ELISA and the concentration determined from standard curves. The average total antibody concentration (IgA, IgG and IgM combined total) in each sample group was calculated and compared. Box plot shows the median at the centre of the box, with the inter-quartile range (IQR) from the upper to lower end of the box. Whiskers are plotted at 1.5 times the IQR from the end of the box, and values outside this range plotted individually.

FIG. 4: Comparison of the average percentage of specific Ig of the total Ig concentration between sample groups.

IgA, IgG and IgM antibodies were detected in various sample groups by ELISA and the concentration determined from standard curves. The concentration of each antibody isotype was calculated as a percentage of the total antibody measured (IgA, IgG and IgM combined) and the average from each sample group calculated.

FIG. 5: Anti-carbohydrate antibody profiles (IgG, IgM and IgA isotypes) for two healthy volunteers (A and B). Error bars=±SD.

FIG. 6: Anti-carbohydrate antibody profiles (IgG, IgM and IgA isotypes) for a healthy volunteer B taken at n and n+14 weeks. Error bars=±SD.

FIG. 7: Comparison of anti-carbohydrate antibody profiles (IgG, pan IgA, IgM and secretory IgA) in a rectal mucosal sample taken from a patient with colorectal cancer (CRC) with that taken from a non-CRC patient.

### DETAILED DESCRIPTION OF THE INVENTION

In one particular aspect of the invention which may be mentioned, there is provided an in vitro method of diagnosing colorectal cancer in a patient, which comprises the steps of:
(a) quantifying the level of one or more proteins in a sample of colorectal mucosal cells obtained from the patient; and
(b) comparing the level of the one or more proteins quantified in step (a), with the level of the one or more proteins in one or more control samples, such that where the control sample is from a healthy subject, a difference in the level of the one or more proteins is indicative of a diagnosis of colorectal cancer, and where the control sample is from a subject with colorectal cancer, a similarity in the level of one or more proteins is indicative of a diagnosis of colorectal cancer.

In a further particular aspect of the invention which may be mentioned, there is provided an in vitro method of diagnosing colorectal cancer in a patient, comprising:
(a) quantifying the level of one or more proteins in a sample of colorectal mucosal cells obtained from the patient; and
(b) comparing the levels of the one or more proteins in the sample with the amounts present in a sample obtained from the patient on a previous occasion, such that a change in the level of the one or more proteins in the sample is indicative of a diagnosis of colorectal cancer in the patient.

The method described herein can be used to diagnose colorectal cancer which is also commonly known as colon or bowel cancer. Colorectal cancer results from uncontrolled cell growth in the colon, rectum and/or appendix (i.e. parts of the large intestine). Colorectal cancer is a prevalent form of cancer, but it is often difficult to diagnose due to the lack of distinct and early symptoms. It is therefore important to provide a simple and effective diagnostic test to identify patients suffering from colorectal cancer in order to increase their chance of survival.

In an alternative embodiment, the method described herein can be used to diagnose other types of cancer, such as stomach cancer. In a further alternative embodiment, the method can be used to diagnose polyps which are an abnormal growth in the mucosal membrane, commonly found in the mucous membrane of the large intestine. It is particularly important to diagnose polyps, because they may contain the beginnings of a tumor which can lead to colorectal cancer.

In another alternative embodiment, the method described herein can be used to diagnose Inflammatory Bowel Disease (IBD).

In one embodiment, the quantifying step comprises quantifying the level of one or more proteins in a sample obtained from the colorectal mucosa of said patient.

References herein to ‘colorectal mucosa’ refer to the mucosal surface of the colon/rectal muscle wall. Thus, it will be appreciated that the colorectal mucosa is the mucous present on the surface of the colon/rectal muscle wall and said mucous typically comprises cells, DNA and proteins, such as antibodies. Such samples can be obtained using colorectal mucosa sampling devices well known in the art, for example using the cell sampling device described in WO2007/080410.

In one embodiment, the sample of the colorectal mucosa comprises colorectal mucosal cells. References herein to ‘colorectal mucosal cells’ include cells obtained from the surface of the colon/rectal muscle wall.

Existing diagnostic tests (such as those described in WO2010/109196) are performed on serum or plasma samples often obtained from the blood. However, these samples are systemic because the relevant proteins detected to diagnose colorectal cancer are spread throughout the body (i.e. via the blood). This is as opposed to the use of a sample of the colorectal mucosa which contains the relevant proteins produced at the site of the colorectal cancer (i.e. non-systemic). This means the sample is less likely to contain any contaminating proteins from other sites in the body, because the material in the sample has been obtained directly from the colon/rectum.

Mucosal samples are also less likely to contain contaminating proteins from elsewhere in the patient's body because mucosal surfaces coat the “external” surface of the body. This is as opposed to serum/plasma samples which are considered “internal” samples because these fluids come into contact with several internal organs which may shed proteins that will contaminate a sample and affect diagnosis.

In one embodiment, the one or more proteins described herein are antibodies, also known as immunoglobulins (Igs), such as naturally occurring immunoglobulins.

The term “antibody” as used herein includes, but is not limited to: polyclonal, monoclonal, bispecific, humanised or chimeric antibodies, single chain antibodies, Fab fragments and F(ab′)₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule. In addition, the IgGs are classified further into four (human) subclasses with distinct, but related, heavy y chains: IgG1, IgG2, IgG3 and IgG4. Furthermore, the IgAs are classified further into two (human) subclasses with distinct, but related, heavy a chains: IgA1 and IgA2.

In one embodiment, the total level of immunoglobulin is quantified in step (a) of the method described herein, in order to give a total concentration of immunoglobulin. This would allow an appropriate person, such as a clinician, to identify whether the total concentration of immunoglobulin is raised or lowered in a sample, which is then linked to a particular disease state. In one embodiment, the total concentration of immunoglobulin is quantified and an increase compared to a healthy control is indicative of Inflammatory Bowel Disease (IBD).

In an alternative embodiment, the level of each immunoglobulin is quantified in step (a) of the method described herein.

In one embodiment, the one or more immunoglobulins are selected from the group consisting of: IgG, IgM, IgA, IgD and/or IgE. In a further embodiment, the one or more immunoglobulins are selected from: IgG, IgM and/or IgA; such as IgA. In a yet further embodiment, the IgA is secretory IgA (sec IgA). In a yet further alternative embodiment, the IgA is total IgA (also referred to as PAN IgA).

IgA plays a critical role in mucosal immunity and can be found in both monomeric and polymeric forms. Circulating IgA is in monomeric form and secretory IgA consists of at least two IgA molecules joined by a J-protein and secretory component. Secretory IgA is the main immunoglobulin found in mucous secretions, including tears, saliva, colostrum and secretions from the genitourinary tract, gastrointestinal tract, prostate and respiratory epithelium. It is also found in small amounts in blood. The secretory component of the antibody protects it from being degraded by proteolytic enzymes, thus facilitating survival in the harsh gastrointestinal tract environment and provides protection against microbes that multiply in body secretions. The present inventors have surprisingly identified that a decrease in the level of IgA found in colorectal mucosal samples, is indicative of a patient suffering from colorectal cancer.

In one embodiment, the level of IgA, such as the total level of IgA, is quantified and a decrease compared to a healthy control is indicative of colorectal cancer.

In one embodiment, the level of IgG, such as the total level of IgG, is quantified and an increase compared to a healthy control is indicative of colorectal cancer.

In one embodiment, the level of IgM, such as the total level of IgM, is quantified and an increase compared to a healthy control is indicative of IBD. In one embodiment, the level of IgA is quantified and a decrease compared to a healthy control is indicative of IBD. In one embodiment, the level of IgG is quantified and an increase compared to a healthy control is indicative of IBD.

In one embodiment, the level of immunoglobulin(s) is quantified by measuring a signal which results from an immunoglobulin binding to a carbohydrate, such as a carbohydrate-containing antigen.

The term “carbohydrate” is well known in the art, especially the field of biochemistry. It refers to organic compounds which only contain carbon, oxygen and hydrogen atoms, and are typically referred to as “sugars”. These sugar residues are often involved in fundamental processes in living organisms, such as the storage of energy and protein glycosylation.

References herein to “carbohydrate-containing antigen” refer to a compound which contains one or more carbohydrate moieties/sugar residues and an entity which is recognized by an antibody (i.e. an antigen). In most cases, it is the carbohydrate moieties themselves which are recognized by the antibody.

Antibodies reacting with carbohydrate structures occur widely in human sera. The best known naturally occurring anti-carbohydrate antibodies from human sera are those detecting members of the A, B, O, I, and Lewis family of blood group antigens. These human alloantigens are expressed not only in erythrocytes but also in many other cell types and in tissue and secretions throughout the body. The epithelial cells of the colon express glycoproteins and carbohydrates related to the blood group antigens. During neoplastic transformation, the expression of the cell surface glycoproteins can change in a number of ways, including expression of a blood group antigen incompatible with the individual's blood type and expression of glycoconjugates normally expressed in other regions of the gastrointestinal tract. Similar changes in expression have also been noted in pre-malignant polyps.

The modification of expression of carbohydrates in the colon may lead to a modified immune response to those carbohydrates. The present inventors believe that changes in carbohydrate expression in the colon epithelium may lead to a change either in the amount or specificity, of the anti-carbohydrate antibody pool, and that these antibodies may be advantageously detected in colorectal mucosa samples.

In one embodiment, the carbohydrate is selected from the group consisting of: T-antigen, Tn-antigen, Lewis X, alpha-galactosidase (α-gal), Lewis A, Lewis A (lactose spacer), sialyl Lewis X, Lewis X (lactose spacer), blood group H antigen, blood group A antigen, blood group B antigen, Lewis B, Lewis Y or sialyl Lewis A. In a further embodiment, the carbohydrate is selected from the group consisting of: T-antigen, Tn-antigen, Lewis X, alpha-galactosidase (α-gal), Lewis A (lactose spacer), sialyl Lewis X, Lewis X (lactose spacer), blood group H antigen, blood group A antigen, blood group B antigen, Lewis B, Lewis Y or sialyl Lewis A. In a further embodiment, the carbohydrate is selected from the group consisting of: T-antigen, Tn-antigen, Lewis X, α-gal, sialyl Lewis A, Lewis A, Lewis A (lactose spacer), sialyl Lewis X, Lewis X (lactose spacer), Lewis Y, blood group H antigen, blood group A antigen or blood group B antigen. In a further embodiment, the carbohydrate is selected from the group consisting of: T-antigen, Tn-antigen, Lewis X, α-gal, Lewis A (lactose spacer), sialyl Lewis X, Lewis X (lactose spacer), blood group H antigen, blood group A antigen or blood group B antigen. In a yet further embodiment, the carbohydrate is selected from the group consisting of: T-antigen, Tn-antigen, α-gal, blood group H antigen or blood group B antigen. In an alternative embodiment, the carbohydrate is selected from the group consisting of: Lewis A, Lewis Y and sialyl Lewis A.

In a further embodiment, the carbohydrate is selected from the group consisting of: T-antigen, Tn-antigen, Lewis X, α-gal, Lewis A (lactose spacer), sialyl Lewis X, Lewis X (lactose spacer), blood group H antigen, blood group A antigen, blood group B antigen, Lewis A, Lewis Y and sialyl Lewis A.

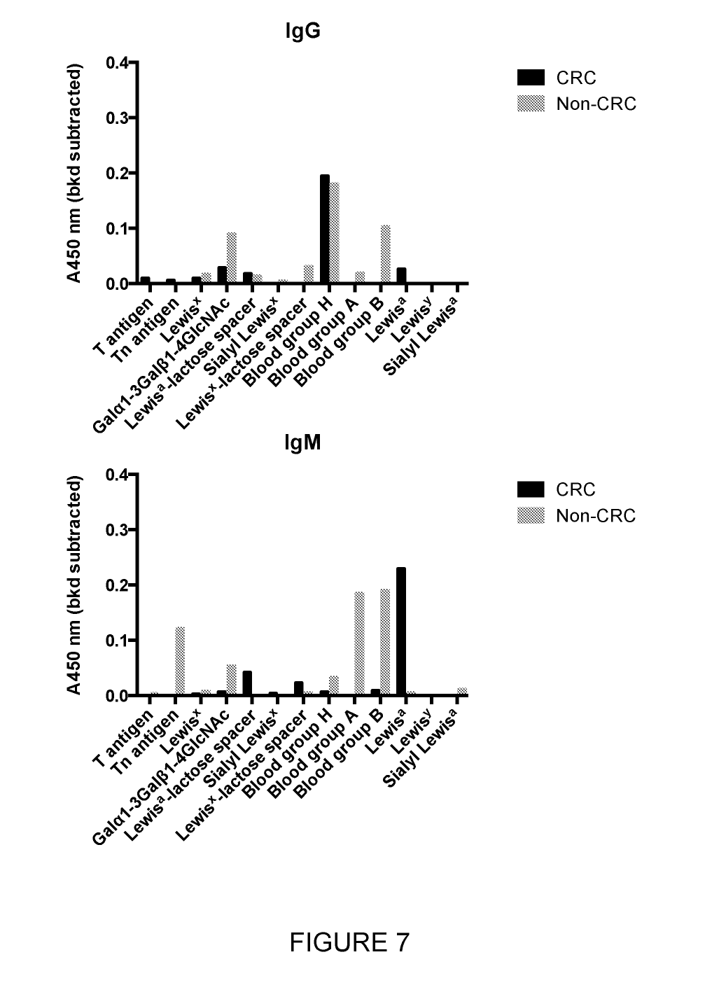

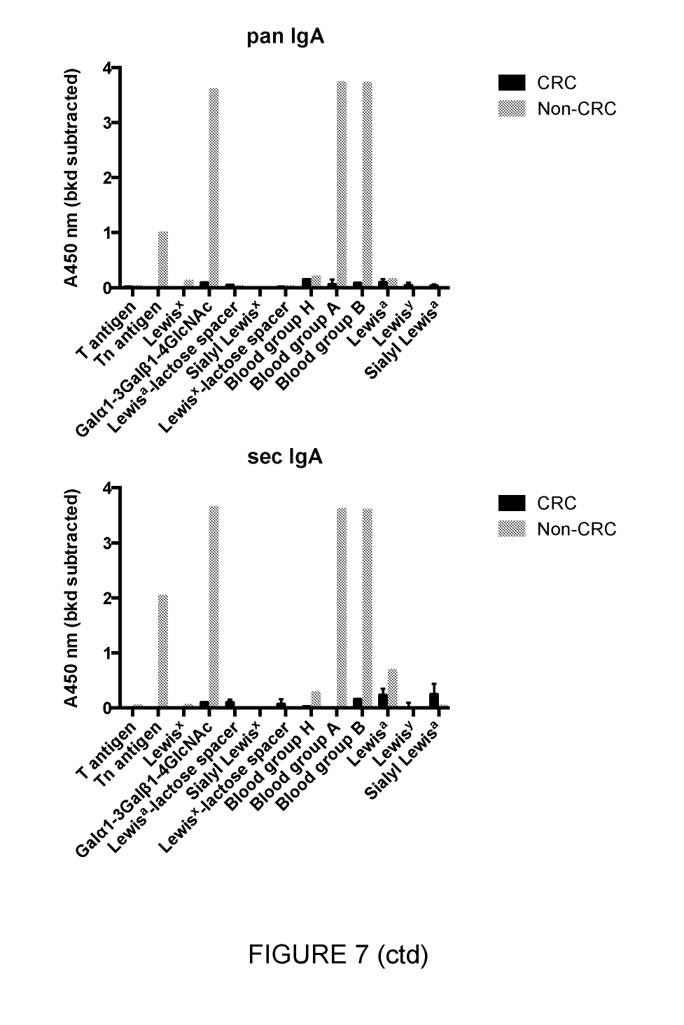

In a yet further embodiment, the carbohydrate is selected from the group consisting of: Tn antigen, α-gal, blood group A antigen or blood group B antigen. The carbohydrates of this embodiment provided excellent results in differentiating between a colorectal patient and a non-colorectal patient using pan IgA and sec IgA antibodies as shown in FIG. 7.

In a yet further embodiment, the carbohydrate is blood group B antigen.

In one embodiment, the level of immunoglobulin binding to T-antigen, Tn-antigen, Lewis X, α-gal, Lewis A (lactose spacer), sialyl Lewis X, Lewis X (lactose spacer), blood group H antigen, blood group A antigen, blood group B antigen, Lewis B, Lewis Y and sialyl Lewis A is quantified. In a further embodiment, the level of immunoglobulin binding to T-antigen, Tn-antigen, Lewis X, α-gal, Lewis A (lactose spacer), sialyl Lewis X, Lewis X (lactose spacer), blood group H antigen, blood group A antigen and blood group B antigen is quantified. In a yet further embodiment, the level of immunoglobulin binding to T-antigen, Tn-antigen, α-gal, blood group H antigen and blood group B antigen is quantified.

In one embodiment, the level of immunoglobulin binding to one or more of T-antigen, Tn-antigen, α-gal, blood group H antigen and/or blood group B antigen is quantified and a decrease compared to a healthy control is indicative of colorectal cancer. In one embodiment, the level of immunoglobulin binding to blood group B antigen is quantified and a decrease compared to a healthy control is indicative of colorectal cancer. In one embodiment, the level of immunoglobulins binding to T-antigen, Tn-antigen, α-gal, blood group H antigen and blood group B antigen is quantified and a decrease compared to a healthy control is indicative of colorectal cancer.

The quantified level of the one or more proteins, in particular more than one protein, may produce a pattern or profile of levels that represents a “fingerprint” for the tested colorectal mucosal sample. This pattern or “fingerprint” can then be compared with equivalent results obtained from healthy controls and/or controls with a particular disease state, so that a diagnosis can be determined by identifying whether a test sample is similar to a healthy control or a control with a particular disease state.

It will be understood that an individual will be indicated to be ‘healthy’, if the level of the one or more proteins in a sample from the individual is similar to a healthy control profile and different to a diseased profile. However, an individual will be indicated to be suffering from a particular disease, if the level of the one or more proteins in a sample from the individual is different to a healthy control profile and similar to a selected disease profile.

The level of one or more proteins in one or more “controls” used in the method of the invention may be provided as a reference value for the expression level of the chosen protein in a healthy test subject, and/or a test subject with a particular disease state, such as a subject with colorectal cancer. It may be understood that a control sample may include a sample previously taken from the same patient. A reference value may be devised from a statistical assessment of the expression levels of a particular protein, generated from biological samples taken from a plurality or statistically-significant number of healthy or test subjects with colorectal cancer.

The levels of one or more proteins in a sample can be compared with the levels of one or more proteins in a sample obtained from the same patient on a previous occasion, such that any changes in the profile may be used as an early indicator of colorectal cancer or may be used to monitor the progression of the disease.

References herein to a “difference” in the level of one or more proteins in a test sample, as opposed to a control, suggests that there is a statistically significant change in the level of the one or more proteins. However, it will be appreciated that where the control is a subject with colorectal cancer, the method of the invention will require a statistically similar result to provide a positive diagnosis of colorectal cancer. In one embodiment, an increase in the level of a protein is indicative that the patient has colorectal cancer. In an alternative embodiment, a decrease in the level of a protein is indicative that the patient has colorectal cancer.

References herein to a “similarity” in the level of one or more proteins in a test is sample, when compared to a control, suggests that there is no statistically significant difference in the level of the one or more proteins.

The term “diagnosis” as used herein encompasses identification, confirmation, and/or characterisation of colorectal cancer. Methods of monitoring and of diagnosis according to the invention are useful to confirm the existence of a disorder; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects.

In a further particular aspect of the invention which may be mentioned, there is provided an in vitro method of monitoring efficacy of a therapy for colorectal cancer in a patient having such a disorder or suspected of having such a disorder, comprising:
(a) quantifying the level of one or more proteins in a sample of colorectal mucosal cells obtained from the patient; and
(b) comparing the levels of the one or more proteins in the sample with the amounts present in a sample obtained from the patient on a previous occasion, such as prior to commencement of therapy, such that a difference in the level of the one or more proteins in the sample is indicative of a beneficial effect of the therapy.

In one embodiment the level of IgA is quantified and an increase compared to a sample obtained from the patient on a previous occasion is indicative of a beneficial effect of the therapy. In one embodiment, the level of IgG is quantified and a decrease compared to a sample obtained from the patient on a previous occasion is indicative of a beneficial effect of the therapy.

In one embodiment, the level of immunoglobulin binding to one or more of T-antigen, Tn-antigen, α-gal, blood group H antigen and/or blood group B antigen is quantified and an increase compared to a sample obtained from the patient on a previous occasion is indicative of a beneficial effect of the therapy. In one embodiment, the level of immunoglobulin binding to blood group B antigen is quantified and an increase compared to a sample obtained from the patient on a previous occasion is indicative of a beneficial effect of the therapy. In one embodiment, the level of immunoglobulins binding to T-antigen, Tn-antigen, α-gal, blood group H antigen and blood group B antigen is quantified and a decrease compared to a sample obtained from the patient on a previous occasion is indicative of a beneficial effect of the therapy.

In monitoring methods, test samples may be taken on two or more occasions. The method may further comprise comparing the level of the one or more proteins present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the one or more proteins in test samples taken on different occasions.

Quantifying the amount of the one or more proteins present in a sample may include determining the concentration of the one or more proteins present in the sample. Detecting and/or quantifying may be performed directly on the sample, or indirectly on an extract therefrom, or on a dilution thereof.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the one or more proteins in the sample or samples. The samples can be prepared, for example where appropriate diluted or concentrated, and stored in the usual manner.

Detection and/or quantification of one or more proteins may be performed by detection of the one or more proteins or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

Detecting and/or quantifying the one or more proteins may be performed using an immunological method, involving an antibody, or a fragment thereof capable of specific binding to the target proteins. Suitable immunological methods include sandwich immunoassays, such as sandwich ELISA; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the one or more proteins.

In immunonephelometry, the interaction of an antibody and target antigen on the one or more proteins results in the formation of immune complexes that are too small to precipitate. However, these complexes will scatter incident light and this can be measured using a nephelometer. The antigen concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA).

ELISA is a biochemical technique used for detecting and quantifying substances such as an antibody or an antigen in a sample. One of the types of ELISA is called a “sandwich” assay because the substance to be measured is bound between two antibodies—the capture and the detection antibody. This format of ELISA is often preferred because it is sensitive and robust. When designing sandwich ELISA it is important that the capture and the detection antibodies must recognise two separate non-overlapping epitopes. Capture and detection antibodies that do not interfere with one another and can bind simultaneously to the antigen are considered a matched pair and are suitable for developing a sandwich ELISA. For accurate quantitative results, the signal of the unknown substance of interest should always be compared against those of standards included on the same ELISA plate. The concentration range of the standards should be optimized to ensure a suitable standard curve.

In one embodiment, the level of one or more proteins is quantified using an ELISA technique. In one embodiment, the carbohydrate(s), such as the carbohydrate containing antigen(s), are coated onto the substrate or surface, and the sample is applied to the coated substrate. After the antibodies in a sample have bound and the unbound antibodies have been washed away, the standard ELISA protocol is followed in order to detect the amount of bound antibody.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of the one or more proteins of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc.) is labelled with biotin and the other partner (surface, e.g. well, bead, sensor etc.) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for a target protein of the invention may be immobilised on an avidin or streptavidin surface, the immobilised ligand may then be exposed to a sample containing or suspected of containing the one or more proteins in order to detect and/or quantify one or more proteins of the invention. Detection and/or quantification of the immobilised antigen may then be performed by an immunological method as described herein.

The one or more proteins may be directly detected, e.g. by SELDI or MALDI-TOF. Alternatively, the one or more proteins may be detected directly or indirectly via interaction with a ligand or ligands such as an antibody or a target protein-binding fragment thereof, or other peptide, or ligand, e.g. aptamer, or oligonucleotide, capable of specifically binding the one or more proteins. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving detection and/or quantification of the one or more proteins of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

In one embodiment, detecting and/or quantifying is performed using a biosensor or a microanalytical, microseparation or immunochromatography system. In a further embodiment, the biosensor is an acoustic, plasmon resonance, holographic or microengineered sensor.

Methods of the invention can be performed in array format, e.g. on a chip, or as a multi-well array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

Diagnostic or monitoring kits are provided for performing methods of the invention. Such kits will suitably comprise a ligand for detection and/or quantification of the one or more proteins, and/or a biosensor, and/or an array as described herein, optionally together with instructions for use of the kit.

In a further particular aspect of the invention which may be mentioned, there is provided a kit for use in the methods defined herein, comprising a biosensor capable of detecting and/or quantifying the one or more proteins, in a colorectal mucosal cell sample.

In one embodiment, the kit further comprises one or more carbohydrates, such as carbohydrate-containing antigens, as defined herein.

Suitably a kit according to the invention may contain one or more components selected from the group: a ligand specific for the one or more proteins or a structural/shape mimic of the one or more proteins, one or more controls, one or more reagents and one or more consumables; optionally together with instructions for use of the kit in accordance with any of the methods defined herein.

In one embodiment, the kit comprises one or more carbohydrates according to the method of the present invention, immobilized on a suitable substrate, such as a microtitre plate well, together with a reagent capable to detect an antibody bound to the plate well. The kit may contain additional reagents, such as wash solutions, solutions for the dilution of samples, enzyme substrates and other reagents common to ELISA kits.

In one embodiment, the kit may include reagents which measure the level of one or more proteins binding to more than one carbohydrate. The kit may be provided with more than one type of carbohydrate coated onto multiple, separate substrates (i.e. multiple wells on a microtitre plate).

A biosensor, as described herein, is an analytical device for the detection of an analyte (such as the one or more proteins described herein) that combines a biological component with a detector. As used herein, the term “biosensor” means anything capable of detecting and/or quantifying the one or more proteins. Examples of biosensors are described herein.

Biosensors according to the invention may comprise a ligand or ligands capable of specific binding to the one or more proteins. Such biosensors are useful in detecting and/or quantifying the one or more proteins.

The one or more proteins detected in the method of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the target protein. On exposure, the target protein reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular protein of interest is feasible.

Suitably, biosensors for detection of the one or more proteins of the invention combine biomolecular recognition with appropriate means to convert detection of the presence, or quantitation, of the protein(s) in the sample into a signal. Biosensors can be adapted for “alternate site” diagnostic testing, e.g. in the ward, outpatients' department, surgery, home, field and workplace.

Biosensors to detect the one or more proteins of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more proteins of the invention.

The biosensor may incorporate an immunological method for detection of the one or more proteins, electrical, thermal, magnetic, optical (e.g. hologram) or acoustic technologies. Using such biosensors, it is possible to detect the target proteins at the anticipated concentrations found in biological samples.

In a further embodiment, the kit may further comprise a device for obtaining the sample of the colorectal mucosa, for instance the colorectal mucosal sampling device described in WO2007/080410.

Kits are also vital as patient monitoring tools, to enable the physician to determine whether relapse is due to worsening of the disorder. If treatment is assessed to be inadequate, then therapy can be modified, reinstated or increased; a change in therapy can be given if appropriate. As the method described herein is sensitive to the state of the disorder, it may be used to provide an indication of the impact of a particular therapy.

The following studies illustrate the invention:

### EXAMPLE 1

### Detecting Total Concentration of Antibodies in Clinical Samples Using an Anti-Human Antibody ELISA

Materials and Methods

Reagents

| | | Stock |
|---|---|---|
| Component | Supplier | concentration |
| Carbonate-bicarbonate buffer tablets | Sigma | 1 tab = 100 ml |
| Phosphate buffered saline (PBS) | Fisher Scientific | 10x |
| Tween 20 | Fisher Scientific | 1x |
| BSA | Sigma | 1x |
| MAb anti-human IgM Fc | Stratech (HRL) | 2 mg/ml |
| MAb anti-human IgA Fc PAN | Stratech (HRL) | 2 mg/ml |
| MAb anti-human IgG Fc | Stratech (HRL) | 2 mg/ml |
| MAb anti-human SecIgA - biotin | Stratech (HRL) | 1 mg/ml |
| MAb anti-human IgA Fab PAN - | Stratech (HRL) | 1 mg/ml |
| biotin | | |
| MAb anti-human IgG Fd - biotin | Stratech (HRL) | 1 mg/ml |
| MAb anti-human IgM Fc - biotin | Stratech (HRL) | 1 mg/ml |
| IgA from human colostrums | MP Biomedicals | 5 mg/ml |
| IgM from human serum | Sigma | 0.8 mg/ml |
| IgG from human serum | Sigma | 6 mg/ml |
| ExtrAvidin ™-Peroxidase | Sigma | 1.23 mg/ml |
| Enhanced K-Blue TMB Substrate | Skybio | 1x |
| HCl | Fisher Scientific | 36% (11.65M) |

Buffers

| Buffer | Composition |
|---|---|
| Coat buffer | 50 mM Na₂CO₃, pH 9.6 |
| PBS | 11.9 mM Na₂HPO₄, 11.9 mM KH₂PO₄, 137 mM |
| | NaCl, 2.7 mM KCl |
| Wash buffer | PBS + 0.05% Tween-20 |
| Block buffer | PBS + 0.5% (v/v) Tween-20 + 1% (w/v) BSA |
| Stop solution | 2M HCl |

Clinical Samples

The colorectal mucosal samples used in the ELISA assay were collected during a clinical study performed in University College London Hospital (UCLH). In order to preserve any antibodies present, samples were collected in a buffer which protects proteins from proteolytic degradation. The buffer works by precipitating all of the proteins within a sample. Precipitated proteins are stable and biologically inactive, and thus cannot be degraded by proteolytic enzymes in the same sample. Upon arrival at the laboratory, the samples were exchanged into TBS, 1% Titron X, then incubated in lysis buffer for 1 hour prior. Aliquots of 0.5 ml were prepared, snap frozen and then stored at −20° C.

Protocol
1. Prepare coating buffer by emptying 1 tablet in 100 ml of deionised water and mix until completely dissolved.
2. Prepare 5 μg/ml solutions of anti-IgA, anti-IgG and anti-IgM capture antibodies in coating buffer.
3. Load 100 μl of solution into each well and incubate at 4° C. overnight.
4. Wash 4 times with 250 μl/well of wash buffer:
5. Load 300 μl of block buffer into each well and incubate for 1 hour at room temperature.
6. Wash 4 times as per step 3.
7. Load 100 μl of purified IgM, IgA and IgG antibody solutions (in block buffer) into each well and dilutions of the clinical samples and incubate for 1 hour at room temperature.
8. Wash 4 times as per step 3.
9. Load 100 μl/well of detection antibody solution (in block buffer) and incubate for 1 hour at room temperature.
10. Wash 4 times as per step 3.
11. Load 100 μl/well of ExtrAvidin™ peroxidase and incubate for 1 hour at room temperature.
12. Wash 4 times as per step 3.
13. Load 100 μl/well of K-blue TMB substrate to all wells and incubate for approximately 2.5 minutes. Gently swirl the plate during this step.
14. Add 25 μl/well 2M HCl to all wells to stop the enzyme reaction.
15. Read on a plate reader at A450 nm.

Results

Assay Development

The first stage of the experiment described herein involved development of an Enzyme-linked Immunosorbant Assay (ELISA), to specifically detect the human antibody isotypes IgA, IgG and IgM.

In order to detect antibodies in the clinical samples, matched pairs of capture and detection monoclonal antibodies against purified human IgG, IgM, secretory IgA (secIgA) and PAN IgA (i.e. total IgA) were identified and tested. To be able to measure accurately the amounts of human antibodies potentially present in clinical samples, a standard curve using known amounts of purified human antibodies: IgG, IgM and IgA was optimised.

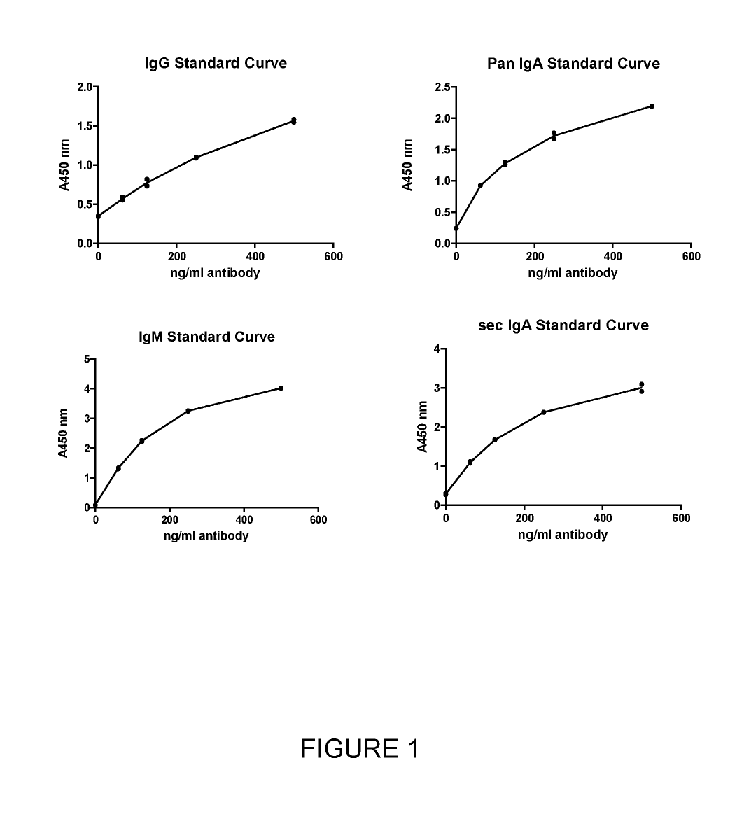

The optimisation steps included varying capture and detection antibodies, concentrations, testing different clones of anti-human secIgA antibody and testing various sources of human secretory IgA antibody. An optimised standard curve for each matched pair of antibodies against human IgG, IgM, secIgA and PAN IgA is shown in FIG. 1.

Results of Assay Development

Two samples were tested for the presence of IgA, IgG and IgM antibodies using the technique described above. The colonoscopy results for the patient from which the samples were taken revealed no clinical abnormalities. FIG. 1 shows the standard curves for each of the antibody isotypes. These were used to estimate the concentration of antibodies in the sample.

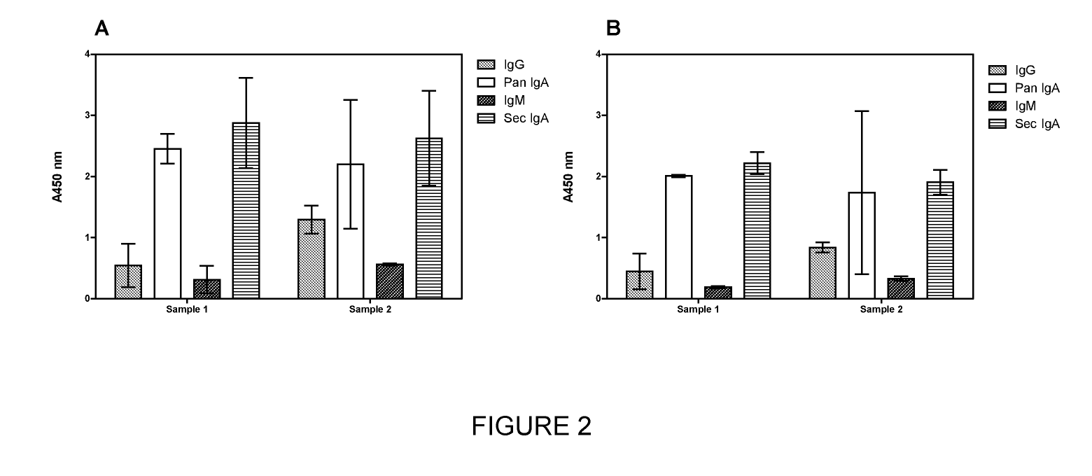

All antibody isotypes, except IgM, were found to be present in the clinical samples. FIG. 2 shows the signals obtained from the sample used 50× and 100× dilution (the latter allows the concentration to be estimated from the standard curves). As expected, IgA represents the highest antibody fraction.

TABLE 1
Estimated antibody concentrations in a sample taken from
human colorectal mucosa

| Antibody | | PAN IgA | IgG | sec IgA | IgM |
|---|---|---|---|---|---|
| Concentration | Sample 1 | 40 | 1 | 29 | Not |
| (μg/ml) | | | | | detectable |
| | Sample 2 | 32 | 17 | 23 | Not |
| | | | | | detectable |

The results showed the ELISA technique developed as described, successfully detected the presence of antibodies in samples taken from human colorectal mucosa.

Results of Measuring Total Antibody Concentration

Thirty samples of colorectal mucosa collected in protein preservation buffer were screened for the presence of antibodies. The sample cohort comprised samples from patients diagnosed with Colorectal Cancer (CRC) (n=5), Inflammatory Bowel Disease (IBD) (n=3), Polyps (n=8), Diverticular disease (n=4), Other disease (n=4) and No apparent disease (NAD) (n=4).

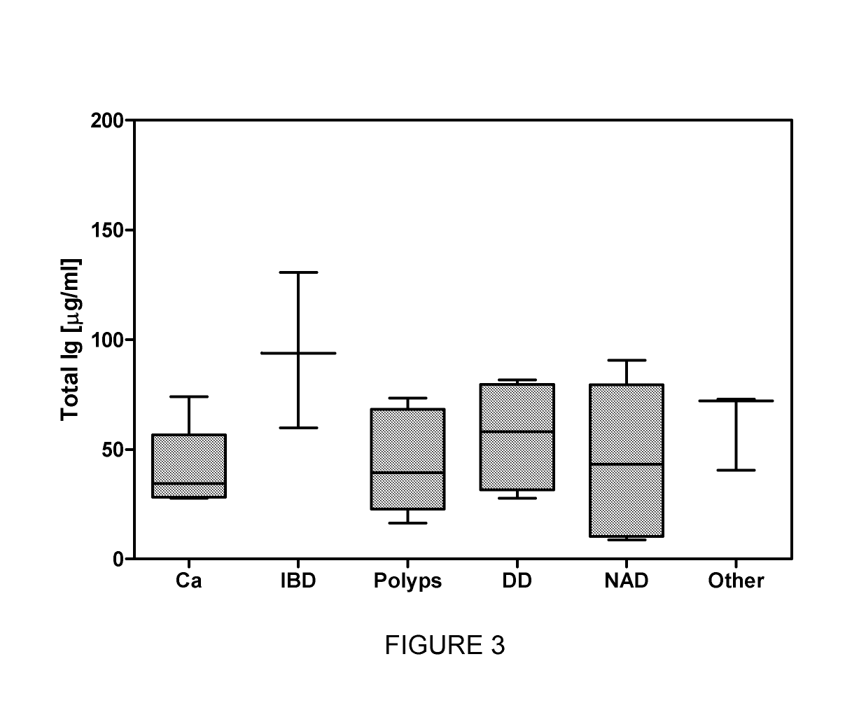

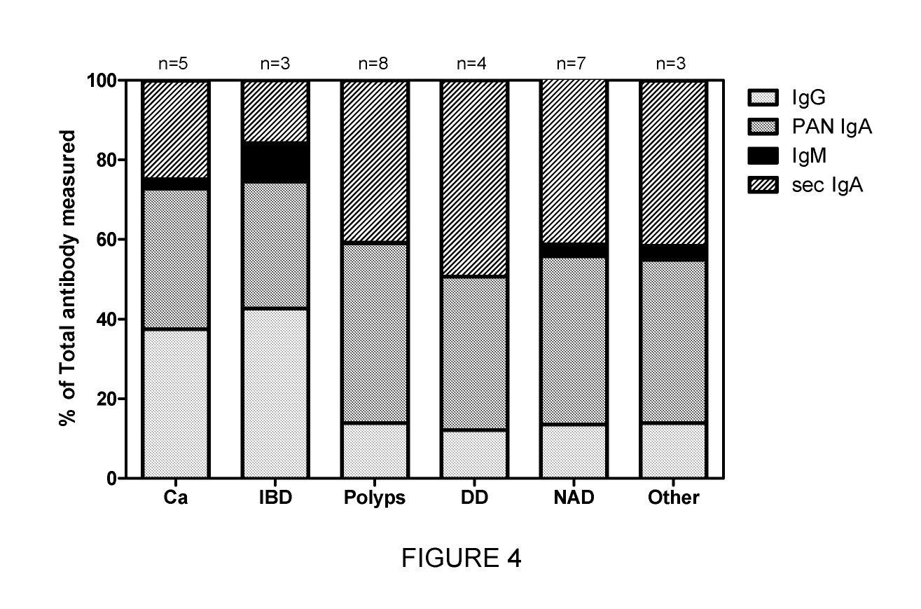

The average total amount of antibody measured in the samples was fairly consistent, although the data from IBD patients indicated a higher average amount. However, with the small number of samples (n=3) and wide 95% confidence intervals, no sound conclusion could be made (FIG. 3).

The data was then examined to see if there were any differences in the amount of specific Ig across the different sample groups. This comparison was made by assessing the average amount of each antibody isotype expressed as a percentage of the average total antibody measured (FIG. 4).

Although numbers were small, there appeared to be differences in the antibody profiles of samples taken from patients with cancer or IBD compared to those with non-serious or no bowel disease. The IBD samples contain a larger proportion of IgM antibody compared to the cancer group, which most probably accounts for the increased total amount of antibody measured in the IBD samples. Whilst the cancer samples contain a very small amount of IgM, the main difference when compared to the other groups (excluding IBD) is a change in the relative amounts of antibody rather than the total amount.

Conclusion

The work presented herein successfully demonstrates the presence of antibodies in the colorectal mucosal samples. In addition, the data highlights potential differences in the antibody profiles of samples from patients with different disease states.

### EXAMPLE 2

### Anti-Carbohydrate ELISA with IgA Detection

Materials and methods

Reagents

| Component | Supplier | Stock concentration | |
|---|---|---|---|
| Carbonate-bicarbonate | Sigma | 1 | tab = 100 ml |
| buffer tablets | | | |
| LNFP II-BSA/Lewis^{a}- | Dextra Laboratories | 1 | mg/ml in DPBS |
| BSA (lactose spacer) | | | |
| LNFP III-BSA/LewisX- | Dextra Laboratories | 1 | mg/ml in DPBS |
| BSA (lactose spacer) | | | |
| LNFP I-BSA/Blood | Dextra Laboratories | 1 | mg/ml in DPBS |
| group H TI | | | |
| T-antigen-HSA | Dextra Laboratories | 1 | mg/ml in DPBS |
| Tn-antigen-HSA | Dextra Laboratories | 1 | mg/ml in DPBS |
| Lewis^{x}-BSA | Dextra Laboratories | 1 | mg/ml in DPBS |
| 3′-Sialyl Lewis^{x}-BSA | Dextra Laboratories | 1 | mg/ml in DPBS |
| Blood group A-BSA | Dextra Laboratories | 1 | mg/ml in DPBS |
| Blood group B-BSA | Dextra Laboratories | 1 | mg/ml in DPBS |
| Galα1-3Galβ1- | Dextra Laboratories | 1 | mg/ml in DPBS |
| 4GlcNAc-BSA | | | |
| BSA (coat) | Sigma-Aldrich | N/A | |
| HSA (coat) | Sigma-Aldrich | N/A | |
| BSA (block) | Sigma-Aldrich | N/A | |
| Tween 20 | Fisher Scientific | 1x | |
| Phosphate buffered saline | Fisher Scientific | 10x | |
| (PBS) | | | |
| MAb anti-human | Stratech (HRL) | 1 | mg/ml |
| SecIgA - biotin | | | |
| ExtrAvidin ™- | Sigma | 1.23 | mg/ml |
| Peroxidase | | | |

| | | |
|---|---|---|
| Enhanced K-Blue TMB | Skybio | 1x |
| Substrate | | |
| HCl | Fisher Scientific | 36% (11.65M) |
| 96 well high binding half | Griener Bio-one | N/A |
| area plate | | |

Buffers

Same as described in Example 1, above.

Protocol
1. Prepare 5 μg/ml of each antigen, 5 μg/ml BSA (coat), 5 μg/ml HSA in coat buffer.
2. Coat plate with 70 μl per well and incubate overnight at 4° C.
3. Wash 3×150 μl/well of wash buffer.
4. Add 150 μl/well of block buffer and incubate for 1 hour at room temperature.
5. Wash 3×150 μl/well of wash buffer as described in step 3.
6. Load 70 μl of 1/10 dilution of samples (in block buffer). Incubate for 2 hours at room temperature.
7. Wash 3×150 μl/well of wash buffer as described in step 3.
8. Prepare dilution of the detection antibody in block buffer and add 70 μl/well (requires 7.5 ml per plate). Incubate for 1 hour at room temperature.
9. Wash 3×150 μl/well of wash buffer as described in step 3.
10. Prepare a 1:1000 dilution of ExtrAvidin™ peroxidase by adding 7.5 μl of stock to 6992.5 μl of block buffer. Add 70 μl per well and incubate for 1 hour at room temperature.
11. Wash 3×150 μl/well of wash buffer as described in step 3.
12. Load 70 μl/well of K-blue TMB substrate to all wells and incubate for 15 minutes. Gently swirl the plate during this step.
13. Add 20 μl/well 2M HCl to all wells to stop the enzyme reaction.
14. Read on a plate reader at A450 nm.

Results

91 samples collected from patients with a positive Fecal Occult Blood Test (FOBT) were tested for anti-IgA antibodies to 10 carbohydrate antigens (T-antigen, Tn-antigen, Lewis X, α-gal, Lewis A (lactose spacer), sialyl Lewis X, Lewis X (lactose spacer), blood group H antigen, blood group A antigen, blood group B antigen).

TABLE 2
Summary of patient samples collected and analysed

| Disease | No. of samples |
|---|---|
| Colorectal Cancer | 6 |
| Diverticulosis | 12 |
| Diverticulosis/Polyps | 6 |
| Haemorrhoids | 7 |
| IBD | 3 |
| No associated disease | 28 |
| Polyps | 29 |

Mann-Whitney tests were used to compare ELISA scores between diseased and non-diseased subjects for each carbohydrate antigen. ‘Diseased’ was defined in three different ways: colorectal cancer, colorectal cancer and/or polyps, any abnormality. There was significant association between colorectal cancer and ELISA for T-antigen, Tn-antigen, α-gal, blood group H antigen, blood group B antigen. Therefore, statistical analysis performed showed that several antigens have a strong association with disease.

TABLE 3
Association of anti-carbohydrate antibodies with disease

| | Malignant | Growth | Any |
|---|---|---|---|
| T-antigen | ++ | + | |
| Tn-antigen | ++ | | |
| Lewis X | | | |
| α-gal | ++ | | |
| Lewis A (lactose | | | |
| spacer) | | | |
| Sialyl Lewis X | | | |
| Lewis X (lactose | | | |
| spacer) | | | |
| Blood group H | ++ | | |
| antigen | | | |
| Blood group A | + | + | + |
| antigen | | | |
| Blood group B | +++ | | |
| antigen | | | |

Mann-Whitney tests were used to compare ELISA scores between diseased and non-diseased subjects for each carbohydrate. ‘Diseased’ was defined in three different ways: “malignant” is colorectal cancer, “growth” is any of colorectal cancer or polyps, and “any” is all except no abnormality detected (NAD). Key to statistical significance: “+++”=0.002≦P<0.01, “++”=0.01≦P<0.05 and “+”=0.05≦P<0.2.

Conclusion

The work presented herein successfully demonstrates the presence of anti-carbohydrate antibodies in colorectal mucosal samples. In addition, the data highlights potential differences in the antibody profiles of samples from patients with different colorectal cancer.

### EXAMPLE 3

### Anti-Carbohydrate ELISA with IgA (Pan and Secretory), IgM and IgG with Samples from Healthy Volunteers

Materials and Methods

Reagents

Same as described in Example 2, above, with the addition of the following detection antibodies:

| Component | Supplier | Stock concentration |
|---|---|---|
| Mab anti-human PANIgA | Stratech (HRL) | 1 mg/ml |
| Mab anti-human IgM | Stratech (HRL) | 1 mg/ml |
| Mab anti-human IgG | Stratech (HRL) | 1 mg/ml |

Buffers

Same as described in Example 1, above.

Samples

The colorectal mucosal samples were taken from two healthy volunteers (volunteer (a) and volunteer (b)). A second sample was taken from volunteer (b) 14 weeks after the first sample and the results compared.

Protocol

Same as described in Example 2, above.

Results

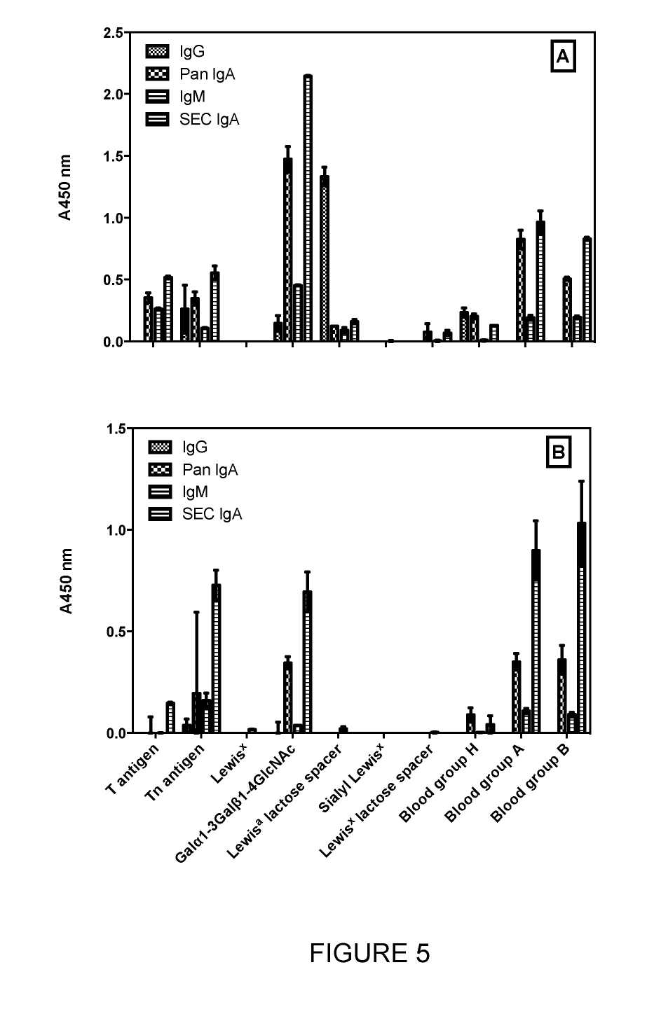

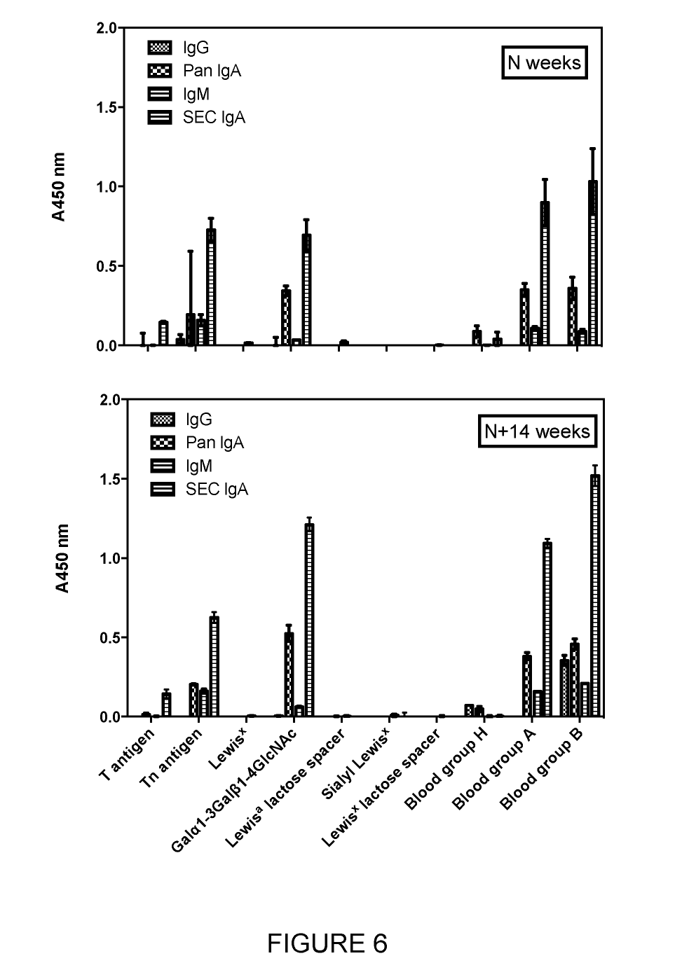

A sample was collected from two healthy volunteers and tested for anti-IgA, anti-IgM and anti-IgG antibodies to 10 carbohydrate antigens (T-antigen, Tn-antigen, Lewis X, α-gal, Lewis A (lactose spacer), sialyl Lewis X, Lewis X (lactose spacer), blood group H antigen, blood group A antigen, blood group B antigen). The results demonstrate that anti-carbohydrate antibodies of all four isotypes are present in the rectal mucosa of healthy individuals (FIG. 5). In addition, the second sample taken from volunteer (b) indicates that the signal is stable over time (FIG. 6).

### EXAMPLE 4

### Anti-Carbohydrate ELISA with IgA Using Additional Carbohydrate Antigens

Materials and Methods

Reagents

Same as described in Example 2, above, with the following carbohydrates:

| Component | Supplier | Stock concentration |
|---|---|---|
| Lewis^{a}-BSA | Dextra Laboratories | 1 mg/ml in DPBS |
| Lewis^{y}-BSA | Dextra Laboratories | 1 mg/ml in DPBS |
| Sialyl Lewis^{a}- BSA | Dextra Laboratories | 1 mg/ml in DPBS |

Buffers

Same as described in Example 1, above.

Samples

Same as described in Example 2, above, but samples had been stored at −80° C. for 18 months.

Protocol

Same as described in Example 2, above.

Results

Mann-Whitney tests were used to compare ELISA scores between diseased and non-diseased subjects for each carbohydrate antigen. ‘Diseased’ was defined in three different ways: “malignant” is colorectal cancer, “growth” is any of colorectal cancer or polyps, and “any” is all except no abnormality detected (NAD). Key to statistical significance: “+++”=0.002≦P<0.01, “++”=0.01≦P<0.05 and “+”=0.05≦P<0.2. The analysis demonstrated a weak association of the measurement of IgA antibodies to sialyl Lewis^{a} with CRC/polyps.

TABLE 4
Association of anti-carbohydrate antibodies with disease

| | Malignant | Growth | Any |
|---|---|---|---|
| Lewis^{a} | | | |
| Lewis^{y} | | | |
| Sialyl Lewis^{a} | | + | + |

### EXAMPLE 5

### Anti-Carbohydrate ELISA with IgA (Pan and Secretory), IgM and IgG with a Sample from a Colorectal Cancer (CRC) Patient and a Sample from a Non-CRC Patient

Materials and Methods

Reagents

Same as described in Example 2 above.

Buffers

Same as described in Example 2 above.

Samples

The colorectal mucosal samples were taken from a patient with colorectal cancer (confirmed by colonoscopy) and a patient without colorectal cancer (confirmed by colonoscopy).

Protocol

Same as described in Example 2 above, but with the addition of three extra carbohydrate antigens (Lewis^{a}, Lewis^{y} and sialyl Lewis^{a}).

Results

Comparison of the anti-carbohydrate profiles (FIG. 7) indicates that there are major differences in the sample taken from the colorectal cancer patient. Most notably, the dramatic reduction in IgA signals.

1. An in vitro method of diagnosing colorectal cancer in a patient, which comprises the steps of:
(a) quantifying the level of one or more proteins in a sample of the colorectal mucosa obtained from the patient; and
(b) comparing the level of the one or more proteins quantified in step (a), with the level of the one or more proteins in one or more control samples,
such that where the control sample is from a healthy subject, a difference in the level of the one or more proteins is indicative of a diagnosis of colorectal cancer, and where the control sample is from a subject with colorectal cancer, a similarity in the level of one or more proteins is indicative of a diagnosis of colorectal cancer.

2. The in vitro method according to claim 1,
wherein the control sample is a sample obtained from the patient on a previous occasion.

3. The in vitro method according claim 2, wherein the one or more proteins are immunoglobulins selected from the group consisting of IgG, IgM, IgA, IgD and IgE.

4. The in vitro method according to claim 3, wherein the level of each immunoglobulin is quantified in step (a).

5.-6. (canceled)

7. The in vitro method according to claim 3, wherein the level of IgA is quantified and a decrease compared to a healthy control is indicative of a diagnosis of colorectal cancer or the level of IgG is quantified and an increase compared to a healthy control is indicative of a diagnosis of colorectal cancer.

8. (canceled)

9. The in vitro method according to claim 3, wherein the level of immunoglobulin is quantified by measuring a signal which results from an immunoglobulin binding to one or more carbohydrates selected from the group consisting of: T-antigen, Tn-antigen, Lewis X, α-gal, Lewis A (lactose spacer), sialyl Lewis X, Lewis X (lactose spacer), blood group H antigen, blood group A antigen and blood group B antigen.

10.-12. (canceled)

13. The in vitro method according claim 9, wherein the level of immunoglobulins binding to one or more of T-antigen, Tn-antigen, α-gal, blood group H antigen or blood group B antigen is quantified and a decrease compared to a healthy control is indicative of colorectal cancer.

14. An in vitro method of monitoring efficacy of a therapy for colorectal cancer in a patient having such a disorder or suspected of having such a disorder, comprising:
(a) quantifying the level of one or more proteins in a sample of the colorectal mucosa obtained from the patient; and
(b) comparing the levels of the one or more proteins in the sample with the amounts present in a sample obtained from the patient on a previous occasion, such as prior to commencement of therapy, such that a difference in the level of the one or more proteins in the sample is indicative of a beneficial effect of the therapy.

15. The in vitro method according to claim 3, wherein quantifying is performed by measuring the concentration of the one or more proteins in the sample.

16. The in vitro method according to claim 3, wherein detecting and/or quantifying is performed by one or more methods selected from SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique.

17. The in vitro method according to claim 3, wherein the detecting and/or quantifying is performed using a biosensor or a microanalytical, microseparation or immunochromatography system.

18. The in vitro method according to claim 17, wherein the biosensor is an acoustic, plasmon resonance, holographic or microengineered sensor.

19. The in vitro method according to claim 3, wherein quantifying the one or more proteins may be performed using an immunological method selected from the group consisting of radioimmunoassay (RIA), enzyme linked immunosorbent assay (ELISA), enzyme immunoassay (EIA), Fluorescence immunoassay (FIA), western blotting, immunoprecipitation and any particle-based immunoassay.

20.-21. (canceled)

22. The in vitro method according to claim 3, which is conducted on samples taken on two or more occasions from a patient.

23. The in vitro method according to claim 3, further comprising comparing the level of the one or more proteins present in samples taken on two or more occasions.

24. The in vitro method according to claim 14, comprising comparing the amount of the one or more proteins in said test sample with the amount present in one or more samples taken from said subject prior to commencement of therapy, and/or one or more samples taken from said subject at an earlier stage of therapy.

25. The in vitro method according to claim 14, wherein samples are taken prior to and/or during and/or following therapy for colorectal cancer.

26. The in vitro method according to claim 3, further comprising detecting a change in the amount of the one or more proteins in samples taken on two or more occasions.

27. A kit for use in the method according to claim 3, comprising a biosensor capable of detecting and/or quantifying the one or more proteins from the sample of the colorectal mucosa.

28. The kit according to claim 27, which further comprises one or more carbohydrates selected from the group consisting of: T-antigen, Tn-antigen, Lewis X, α-gal, Lewis A (lactose spacer), sialyl Lewis X, Lewis X (lactose spacer), blood group H antigen, blood group A antigen and blood group B antigen.

29.-32. (canceled)
